(19) United States
(12) Patent Application Publication
(10) Pub. No.: US 2003/0144345 A1
(43) Pub. Date: Jul. 31, 2003

(54) Treatment of metabolic diseases with soraphen derivatives

(76) Inventors: Marcel Gubler, Arlesheim (CH); Jacques Mizrahi, Basle (CH)

Correspondence Address: HOFFMANN-LA ROCHE INC., PATENT LAW DEPARTMENT, 340 KINGSLAND STREET, NUTLEY, NJ, 07110

(21) Appl. No.: 10/197,078

(22) Filed: Jul. 17, 2002

(30) Foreign Application Priority Data
Jul. 27, 2001 (EP) 01118316.7

(51) Int. Cl. A61K 31/365

(52) U.S. Cl. 514/450

(57) ABSTRACT

The present invention relates to the use of Soraphen derivatives and pharmaceutically acceptable esters thereof, for the treatment and/or prophylaxis of diseases which are associated with ACCβ activity and/or fatty acid oxidation such as diabetes, obesity and dyslipidemia.

### BACKGROUND OF THE INVENTION

Soraphen derivatives and methods for preparing them had previously been published e.g. in EP 282455 an in EP 358606. Soraphen derivatives were known to be inhibitors of plant Acetyl-Coenzyme-A Carboxylase (ACC). It was previously postulated that ACCβ regulates mitochondrial fatty acid oxidation (Ruderman et al. et al. Am. J. Physiol. 276, E1-E18, 1999) and ACCβ was also linked to various diseases (Abu-Elheiga et al., Science 291, 2613-2616, 2001). However, other known inhibitors of plant ACC possessed no or low effect on fatty acid oxidation.

### SUMMARY OF THE INVENTION

It now unexpectedly has been discovered that Soraphen derivatives of the formula

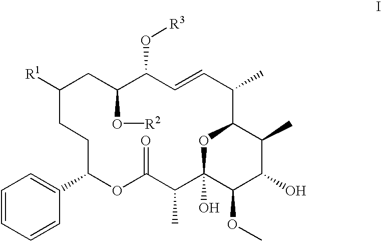

wherein

R₁ is hydrogen or hydroxy,

R² is hydrogen or lower-alkyl,

R³ is hydrogen or lower-alkyl,

and pharmaceutically acceptable esters thereof,

are potent inhibitors of human ACC, ACCα as well as ACCβ, with a novel unknown mode of action that is distinct from simple competitive inhibition such that these derivatives are useful pharmacological agents in the treatment of certain metabolic disorders such as diabetes, obesity and dyslipidemia.

### DETAILED DESCRIPTION OF THE INVENTION

The present invention relates to the use of compounds of formula (I)

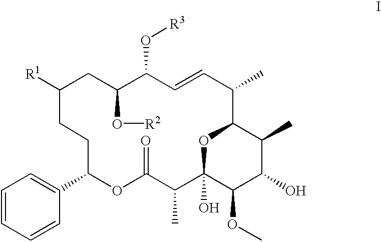

wherein

R¹ is hydrogen or hydroxy,

R² is hydrogen or lower-alkyl,

R³ is hydrogen or lower-alkyl,

and pharmaceutically acceptable esters thereof,

for the preparation of medicaments for the treatment and/or prophylaxis of diseases which are associated with ACCβ and/or fatty acid oxidation, such as diabetes, obesity and dyslipidemia.

It was observed in in-vitro enzyme assays with human ACCβ enzyme that the ACC reaction obeys typical Michaelis-Menten kinetics in dependence on its substrates acetyl-CoA, carbonate and ATP, and that the allosteric activators Na- or K-citrate activate the ACC enzyme in a sigmoidal manner of the Hill-type. Kinetic enzyme assays with different concentrations of Soraphen A 1-α have shown that Soraphen A 1-α does not exhibit a competitive mode of action (i.e. does not change the Km of the substrates acetyl-CoA or ATP) and has little effect on KO 5 of the allosteric activator citrate. Unexpectedly, instead of affecting Kₘ or K_{0.5}, Soraphen A 1-α reduced Vₘₐₓ of the ACC enzyme reaction, indicating that Soraphens do not compete with acetyl-CoA, ATP or citrate for binding to the respective binding sites on the ACC enzyme.

In addition to the inhibitory effect on human ACC, it was further found that Soraphens of formula (I) are highly potent agents to stimulate fatty acid oxidation in liver. Fatty acid oxidation experiments with the well characterized human liver hepatoma cell line HepG2 have shown that the tested Soraphen derivatives at 10 μM stimulated fatty acid oxidation 1.6- to 3.5-fold. In a direct comparison to other known plant-ACC inhibitors, such as e.g. aryloxyphenoxypropionic acids (analogues of herbicides of the Diclofop- and Haloxyfop-type) or cyclohexane-1,3-diones (analogues of herbicides of the cycloxydime- and sethoxidim-type), it was found that the vast majority of such compounds tested in this assay were found to have no stimulatory effect on fatty acid oxidation. Based on the above findings that Soraphens unexpectedly and unlike other known plant-ACC inhibitors stimulate fatty acid oxidation in cultivated human liver hepatoma cells, it is evident that Soraphens have the same stimulatory effect in human liver in vivo.

It was found that Soraphens of formula (I) are highly potent agents to stimulate fatty acid oxidation in muscle, as was shown by means of fatty acid oxidation experiments with cultures of differentiated rat L6 muscle cells. This was confirmed by fatty acid oxidation experiments with cultures of differentiated primary human muscle cells. Based on the above findings that Soraphens stimulate fatty acid oxidation in differentiated rat muscle cells as well as in differentiated primary human muscle cells, it is evident that Soraphens have the same stimulatory effect in human skeletal musce in vivo. Moreover, in vivo experiments in rats confirmed that Soraphens of formula (I) increase fatty acid oxidation and lipid utilization.

These previously unknown and unexpected properties of Soraphen derivatives make these compounds suitable for the use as medicaments, particularly for the treatment and/or prophylaxis of diseases which are related to ACCβ, particularly for the treatment and/or prophylaxis of diseases which are related to reduced rates of fatty acid oxidation such as obesity, dyslipidemias and diabetes.

One application relates to metabolic diseases where low levels of fatty acid oxidation in liver are a problem such as e.g. high fatty acid levels in blood, high triglyceride (TG) levels in blood, dyslipidemias in the form of disturbances in the lipoprotein profile, imbalances in very-low-density lipoprotein (VLDL), low-density lipoprotein (LDL) and high-density lipoprotein (HDL), hepatic overproduction of VLDL-bound TG, and vascular diseases associated with the above metabolic abnormalities, comprising atherosclerosis, hypertension and cardiovascular complications.

Compounds of formula (I) are also useful as medicaments in with the treatment of metabolic complications where low levels of fatty acid oxidation in skeletal muscle are a problem such as high TG levels in muscle, elevated levels of reactive fatty acid esters in muscle such as long chain fatty acyl-CoA, carnitine-CoA and diacylglycerol (DAG), low sensitivity or insensitivity of muscle to the action of insulin due to high TG or elevated levels of reactive fatty acid esters in muscle, impaired glucose tolerance as a consequence of reduced insulin sensitivity, progressing stages of low insulin sensitivity resulting in hyperinsulinemia and insulin resistance, further consequences of insulin resistance such as high blood glucose levels (hyperglycemia) and the development of non-insulin-dependent diabetes mellitus (NIDDM, Type 2 diabetes), further consequences caused by hyperglycemia, e.g. diabetic microvascular diseases in the form of nephropathy, neuropathy, retinopathy and blindness.

Compounds of formula (I) can also be used as medicaments for the treatment of medical indications for which increase in fatty acid oxidation is considered beneficial such as obesity syndromes e.g. excess storage of endogenous lipid (fat), impaired control of appetite and food consumption as a result of low lipid utilization and constant depletion of carbohydrate storage, saving of carbohydrate storage, reduction in the need for carbohydrate supply, suppression of appetite, long term body weight control and maintenance for all persons with genetic, or behavioral inclination to reduced fat oxidation.

Of the diseases mentioned above, the use of compounds of formula (I) as medicaments in context with obesity via increase of mitochondrial fatty acid oxidation in muscle and liver and net increase in energy expenditure in peripheral (muscle) tissue, in context with dyslipidemia via reduced (rebalanced) output by human liver of very low density lipoprotein-bound triglycerides (VLDL-TGs) and in context with insulin resistance and Type II diabetes via reduction in peripheral tissue of high TG levels and reduction of elevated concentrations of the highly reactive esters of fatty acids such as acyl-CoA, carnitine-CoA, diacylglycerol (DAG) are considered to be of particular interest.

The compounds of the present invention further exhibit improved pharmacological properties compared to known ACC inhibitors. The clinical advantage of Soraphens are primarily based on their high potency against ACCβ which is unusual for typical enzyme inhibitors. In addition, since Soraphens exhibit a non-competitive mode of action, their potency is not influenced by changes in metabolite concentrations that can often be observed in different individuals with large variations in metabolic potential or metabolic deficiencies.

Unless otherwise indicated the following definitions are set forth to illustrate and define the meaning and scope of the various terms used to describe the invention herein.

In this specification the term “lower” is used to mean a group consisting of one to seven, preferably of one to four carbon atom(s).

The term “alkyl”, alone or in combination with other groups, refers to a branched or straight-chain monovalent saturated aliphatic hydrocarbon radical of one to twenty carbon atoms, preferably one to sixteen carbon atoms. Alkyl groups can be substituted e.g. with halogen, CN, NO₂, carboxy, and/or aryl. Other, more preferred subsituents are hydroxy, lower-alkoxy, NH₂, N(lower-alkyl)₂, and/or lower-alkoxy-carbonyl. Unsubstituted alkyl groups are preferred.

The term “lower-alkyl”, alone or in combination with other groups, refers to a branched or straight-chain monovalent alkyl radical of one to seven carbon atoms, preferably one to four carbon atoms. This term is further exemplified by such radicals as methyl, ethyl, n-propyl, isopropyl, n-butyl, s-butyl, t-butyl and the like. A lower-alkyl group may have a substitution pattern as described earlier in connection with the term “alkyl”. Unsubstituted lower-alkyl groups are preferred.

The term “pharmaceutically acceptable esters” embraces esters of the compounds of formula (I), in which hydroxy groups have been converted to the corresponding esters with inorganic or organic acids such as sulphuric acid, phosphoric acid, citric acid, formic acid, maleic acid, acetic acid, propionic acid, succinic acid, tartaric acid, methanesulphonic acid, p-toluenesulphonic acid and the like, and amino acids such as glycine, alanine, valine, leucine, iso-leucine and the like, which are non toxic to living organisms.

The compounds of formula (I) in which R¹ is hydrogen or hydroxy are preferred. Further individually preferred compounds of formula (I) are those in which R² is hydrogen, as are those in which R² is methyl. In addition, further individually preferred compounds of formula (I) are those in which R³ is hydrogen, as are those in which R³ is methyl.

A preferred compound of formula I is Soraphen A 1-α, which is a compound of formula Ia:

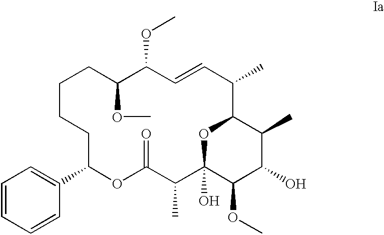

and pharmaceutically acceptable esters thereof.

A further preferred compound of formula (I) is Soraphen A 4-α, which is a compound of formula (I) as described above wherein R¹, R² and R³ are hydrogen. A further preferred embodiment of the present invention thus relates to the use as defined above, wherein the compound of formula (I) is a compound of formula Ib:

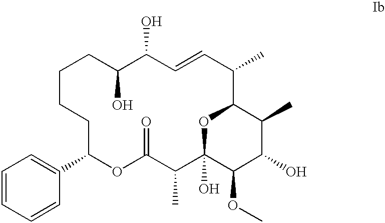

and pharmaceutically acceptable esters thereof.

Another preferred compound of formula (I) is Soraphen B 2-α, which is a compound of formula Ic:

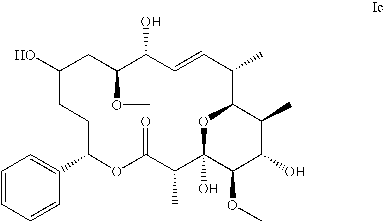

and pharmaceutically acceptable esters thereof.

The invention also relates to pharmaceutical compositions comprising a compound of formula (I), (Ia), (Ib) and/or (Ic) and/or pharmaceutically acceptable esters thereof as defined above, and a pharmaceutically acceptable carrier and/or adjuvant.

In another embodiment, the invention relates to a method for the treatment and/or prophylaxis of diseases which are associated with ACCβ and/or fatty acid oxidation, which method comprises administering a compound of formula (I), (Ia), (Ib) and/or (Ic) and/or pharmaceutically acceptable esters thereof as defined above to a human being or animal. A preferred method as defined above is one, wherein the diesease is diabetes, more preferably non insulin dependent diabetes mellitus. Another preferred embodiment relates to a method as defined above, wherein the disease is obesity. A method as defined above, wherein the disease is dyslipidemia is a further preferred embodiment.

The invention further relates to the use of compounds of formula (I), (Ia), (Ib) and/or (Ic) and/or pharmaceutically acceptable esters thereof as defined above for the treatment and/or prophylaxis of diseases which are associated with ACCβ and/or fatty acid oxidation. In a preferred embodiment, the present invention relates to the use as defined above, wherein the disease is diabetes, preferably non insulin dependent diabetis mellitus. In another preferred embodiment, the invention relates to the use as defined above wherein the disease is obesity. The use as defined above wherein the disease is dyslipidemia is a further preferred embodiment.

In addition, the invention relates to compounds of formula (I), (Ia), (Ib) and/or (Ic) and/or pharmaceutically acceptable esters thereof as defined above for use as therapeutic active substances, particularly as therapeutic active substances for the treatment and/or prophylaxis of diseases which are associated with ACCβ and/or fatty acid oxidation. A preferred embodiment of the present invention relates to compounds for use as therapeutic active substances as defined above, wherein the disease is diabetes, preferably non insulin dependent diabetes mellitus. Another preferred embodiment of the present invention relates to compounds for use as therapeutic active substances as defined above, wherein the disease is obesity. Compounds for use as therapeutic active substances as defined above, wherein the disease is dyslipidemia represent a further preferred embodiment of the present invention.

Compounds of formula (I), (Ia), (Ib) and (Ic) have asymmetric carbon atoms and can exist in the form of optically pure enantiomers or as racemats. It is understood, that enantiomers can exhibit analogous utilities.

Compounds of formula (I), (Ia), (Ib) and (Ic) are preferred over the pharmaceutically acceptable esters thereof. Of the diseases which are associated with ACCβ and/or fatty acid oxidation, those which are associated with fatty acid oxidation are preferred.

It will be appreciated, that the compounds of general formula (I) as well as the compounds of formula (Ia), (Ib) and (Ic) in this invention may be derivatised at functional groups to provide derivatives which are capable of conversion back to the parent compound in vivo.

The conversion of compounds of formula I into pharmaceutically acceptable esters can be carried out by reacting one (or several) of the hydroxyl groups present in a compound of formula (I) with an appropriate carboxylic acid (e.g. acetic acid), using a condensating reagent such as BOP or DCCI, to produce the corresponding pharmaceutically acceptable ester.

The compounds of formula (I), (Ia), (Ib) and (Ic) can be manufactured by methods known in the art, e.g. as described in EP 282455 and EP 358606, or by analogous methods. Compounds of formula (I), (Ia), (Ib), and (Ic) are also commercially available from Gesellschaft für Biotechnologische Forschung mbH (GBF), Braunschweig, Germany.

The following tests were carried out in order to determine the activity of the compounds of formula I.

Production and Characterization of Human ACCβ Enzyme, Its Use in ACC Activity Assays and for Inhibition Studies With Soraphens

The cloning of the full length human muscle-type ACCβ cDNA and expression in HEK293 cells (ATCC, #CRL-1573) was performed as follows. The ACCβ cDNA was amplified by the polymerase chain reaction (PCR) and was cloned using standard recombinant DNA techniques. The PCR reaction was performed with the Expand Long Template PCR System (Roche Molecular Biochemicals, #1 681 8340) and 0.5 ng of cDNA from human skeletal muscle as template. The primers used for PCR amplification were designed on the basis of the published sequence of the human ACCβ cDNA isolated from a human liver cDNA library (Abul-Elheiga et al. J. Biol Chem. 272, 10669-10677, 1997). The sequence of the forward primer ACCB1 was 5′-TTACGCGTGCTAGCCACCATGGTCTTGCTTCTTTGTCTATC-3′; it includes a NheI restriction cleavage site for subcloning and a Kozak translation initiation consensus sequence preceding the ATG start codon. The sequence of the reverse primer ACCB8 was 5′-TTCTCGAGTCAGGTGGAGGCCGGGCTGTC-3′; it includes a stop codon and a XhoI restriction cleavage site for subcloning. The amplified DNA fragment of approximately 7.4 kb was cloned into a mammalian expression vector. The resulting plasmid isolates, pRF33A, B, C, D, and E were individually transfected in human embryonic kidney 293 cells (HEK293) using a standard lipid transfection method. Cell extracts of transfected cells were prepared in a lysis buffer containing 0.4 mg/ml digitonin and enzyme activity was determined using a radiometric ACC activity assay as described below. Plasmid pRF33D gave the highest activity and was chosen for large scale transfections of HEK293 cells and enzyme purification.

Since ACC enzyme activities in crude cell lysates were very low, enrichment of ACCβ enzyme expressed in HEK293 cells was achieved by a single anion exchange chromatography step. Cell lysates were run over a 5 ml Econo Pac High Q column (Bio-Rad, #732-0027). Bound proteins were eluted by a gradient of NaCl from 0 to 1 M in 50 mM Tris-HCl, pH 7.5, 1 mM DTT, 5% glycerol. Fractions containing high ACCβ enzyme activity were pooled and stored at −20° C.

Standard ACCβ enzyme assays, in a total volume of 100 μl, contained 50 mM HEPES-KOH, pH 7.5, 10 mM K-citrate, 10 mM MgSO₄, 1 mM ATP, 0.1 mM DTT, 2% DMSO, 0.1 mg/ml fatty acid-free BSA, 0.2 mM acetyl-CoA, 2 mM KHCO₃, 0.2 mM [¹⁴C]NaHCO₃ (50-60 mCi/mmol) and cell lysate or purified ACCβ enzyme. Reactions were incubated at 37° C. for 45 min. and stopped by the addition of 50 μl of 2 N HCl. Terminated reactions were incubated at 50° C. over night to evaporate non-incorporated [¹⁴C]NaHCO₃. [¹⁴C]-labeled malonyl-CoA reaction product was quantitated by liquid scintillation counting after the addition of 20 μl of Microscint 20 (Canberra Packard, #6013621) on a TopCount NXT microplate scintillation counter (Canberra Packard).

Inhibition of ACCβ activity was determined at saturating substrate concentrations with two-fold serial dilutions of test compounds spanning a concentration range of at least two log units. IC₅₀ values were calculated with the GraFit software (Erithacus Software Ltd.). Kinetic parameters (Kₘ, Vₘₐₓ and K_{0.5}) were determined by varying one substrate (or allosteric effector) in a two-fold serial dilution over a concentration range of at least two log units while keeping all other substrates at constant saturating concentrations. Fitting to the Michaelis-Menten equation (for calculation of Km of substrates) and fitting to the Hill equation (for calculation of K_{0.5}of the allosteric activator K-citrate) was done with the GraFit software. To determine the mode of action of Soraphens, determinations of Kₘ for ATP and acetyl-CoA and of K_{0.5} for K-citrate were repeated in the absence and in the presence of the three different Soraphens at 0.5, 1 and 2 times their IC₅₀ and the data were fitted to the equation for non-competitive inhibition with the GraFit software.

Kinetic analysis of the recombinant human ACCβ enzyme preparation revealed the following kinetic constants: Kₘ values for acetyl-CoA, ATP and NaHCO₃, 0.037 mM, 0.192 mM and 3.5 mM, respectively; K_{0.5} value for K-citrate, 2.5 mM. Titration of the compounds of formula (Ia), (Ib), and (Ic) did not change the Km of acetyl-CoA and ATP and did not change the K_{0.5} of K-citrate but lead to a dose-dependent decrease of Vₘₐₓ in all cases. Therefore, inhibition competitive with acetyl-CoA, ATP or K-citrate can be excluded and a non-competitive mode of action can be postulated. The in vitro activity (IC₅₀ values) of the compounds (Ia), (Ib), and (Ic) against purified ACCβ enzyme were found to be 0.003 uM, 0.2 uM and 1.75 uM, respectively.

The preferred compounds of the present invention exhibit IC₅₀ values of 1 nM to 10 μM, preferrably of 1-200 nM.

Fatty Acid Oxidation Assays With Human Liver Hepatoma Cells

Cultivation of the human liver hepatoma cell line HepG2 for fatty acid oxidation assays was performed as follows. For routine passaging, HepG2 cells (American Type Culture Collection, ATCC) were grown in T75 flasks (Falcon, #353136) in 15 ml of Dulbecco's modified Eagle medium (DMEM, Sigma, #D5796), containing 10% heat inactivated fetal bovine serum (FBS, Summit Biotechnology, #S-100-05) and 1% Penicillin/Streptomycin (Sigma, #P4333). Incubation was done at 37° C. in a humid atmosphere containing 5% CO₂. The cells were passaged once per week by trypsinization and an additional change of medium was done after 3 to 4 days after passaging.

For fatty acid oxidation assays, each well of a 12-well tissue culture plate (Falcon, #353225) was seeded with 1×10⁶ cells in 1 ml of DMEM, 10% FBS, 1% Penicillin/Streptomycin. After 3 days, the medium was replaced by 1 ml of glucose-deficient DMEM (Sigma, #D5030) that was supplemented with 3.7 g/l NaHCO₃ and 25 mM HEPES-NaOH followed by an incubation (starvation) period of 2 to 3 hours at 37° C. In the following, the medium was exchanged again with 0.4 ml of labeling medium that contained [¹⁴C]-labeled palmitatefatty acids as outlined below.

Labeling medium to assay oxidation of fatty acids was composed of glucose-deficient DMEM containing 3.7 g/l NaHCO₃, 25 mM HEPES-NaOH, 0.5% fatty acid-free bovine serum albumin (BSA), 0.1% ethanol, 0.5% DMSO (or test compounds dissolved in DMSO), 5 mM D-glucose, and 16 μM unlabeled palmitate plus 0.25 μM [U-¹⁴C]palmitate (approx. 800 mCi/mmol). After the addition of labeling medium, the cell culture plates were sealed air tight with self adhesive Super Sealing Film (Life Systems Design, #02-2796-3001). Before sealing, twelve 1 cm² squares of filter paper (Schleicher & Schuell, #GB004) pretreated with 50 μl of highly basic tissue solubilizer solution NCSII (Amersham Pharmacia, #NNCS-502) were attached to the lower side of the sealing film. Upon sealing of the tissue culture plates, the filters were positioned in the center above each well, and served to capture the evolving ¹⁴CO₂ that was produced by the cells in each individual well. After an incubation between 3 to 8 hours at 37° C., the filters were removed from the sealing film and the captured ¹⁴CO₂ was quantitated by liquid scintillation counting with 2 ml of scintillation cocktail BCS-NA (Amersham Pharmacia, #NBCS204) in a Tri-Carb liquid scintillation counter (Canberra Packard). Data of fatty acid oxidation assays in Tables 1, 2 and 3 are given as means +/−standard deviation of three independent data poits.

When tested at a fixed concentration of 10 μM on HepG2 cells, compounds (Ia), (Ib) and (Ic) stimulated palmitate oxidation 3.5, 2.8 and 1.6-fold, respecitvely (Table 1). The known hypolipidemic agent TOFA also stimulated palmitate oxidation 2.1-fold when tested at 25 μM. In contrast, the plant ACC inhibitors Diclofop, Haloxyfop and Cycloxidim did not stimulate palmitate oxidation.

TABLE 1
Stimulation of palmitate oxidation rate in HepG2 cells by ACC
inhibitors

| | | Palmitate oxidation rate | Fold |
|---|---|---|---|
| Compound | Conc. (μM) | (pmol · h⁻¹ · well⁻¹) | stimulation^{a} |
| — | — | 3.8 +/− 0.3 | — |
| Cpd (Ia) | 10 | 13.7 +/− 0.7 | 3.5 |
| Cpd (Ib) | 10 | 10.8 +/− 0.4 | 2.8 |
| Cpd (Ic) | 10 | 6.1 +/− 0.4 | 1.6 |
| TOFA^{b} | 25 | 8.1 +/− 0.7 | 2.1 |
| Diclofop^{c} | 25 | 3.1 +/− 1.3 | 0.8 |
| Haloxyfop^{d} | 25 | 3.6 +/− 0.4 | 1.0 |
| Cycloxidim^{e} | 25 | 3.5 +/− 0.2 | 0.9 |

^{a}Fold stimulation over palmitate oxidation rate in the absence of test compounds
^{b}TOFA is 5-(Tetradecyloxy)-2-furoic acid
^{c}Diclofop is 2-[p-(2,4-Dichlorophenoxy)phenoxy]propionic acid
^{d}Haloxyfop is 2-[4-(3-chloro-5-trifluormethyl-2-pyridinyloxy)phenoxy] propionic acid
^{e}Cycloxidim is 2-(1-Ethoxyimino-butyl)-3-hydroxy-5-(tetrahydro-thiopyran-3-yl)-cyclohex-2-enone

Fatty Acid Oxidation Assays With Differentiating Rat Muscle Cells

Cultivation of the differentiating rat muscle cell line L6 for fatty acid oxidation assays was performed as follows. For routine passaging, L6 cells (ATCC) were grown in T75 flasks in 15 ml of MEM Alpha medium (Gibco BRL Life Technologies, #22571-038), containing 10% heat inactivated FBS and 1% antibiotic/antimycotic solution (Gibco BRL Life Technologies, #15240-062). Incubation was done at 37° C. in a humid atmosphere containing 5% CO₂. The cells were passaged twice per week by trypsinization.

For fatty acid oxidation assays, each well of a 12-well tissue culture plate was seeded with 2 to 5×10⁴ cells in 1 ml of MEM Alpha, 10% FBS, 1% antibiotic/antimycotic solution. After 3 days, medium was exchanged by 1 ml of the same, fresh medium. After 5 days, medium was exchanged again, this time by 1 ml of medium containing 2% FBS, 1% antibiotic/antimycotic solution. After 7 days, the cells were fully differentiated and formed large multinucleated myotubes. At this stage, the medium was replaced by 1 ml of glucose-deficient DMEM supplemented with 3.7 g/l NaHCO₃ and 25 mM HEPES-NaOH followed by a starvation period of 2 to 6 hours at 37° C. In the following, the medium was exchanged again with 0.4 ml of labeling medium that contained [¹⁴C]-labeled palmitate fatty acids as outlined for fatty acid oxidation assays with HepG2 cells. The compounds were tested at three different concentrations (2, 0.2 and 0.02 μM) on differentiated L6 cells.

TABLE 2
Stimulation of palmitate oxidation rate in differentiated L6 cells
by inhibitors of ACCβ

| | | Palmitate oxidation rate | Fold |
|---|---|---|---|
| Compound | Conc. (μM) | (pmol · h⁻¹ · well⁻¹) | stimulation^{a} |
| — | — | 9.0 +/− 0.3 | — |
| Cpd (Ia) | 2 | 14.9 +/− 0.4 | 1.7 |
| | 0.2 | 15.8 +/− 0.5 | 1.8 |
| | 0.02 | 16.4 +/− 1.0 | 1.8 |
| Cpd (Ib) | 2 | 13.5 +/− 1.4 | 1.5 |
| | 0.2 | 10.0 +/− 1.4 | 1.1 |
| | 0.02 | 8.8 +/− 0.3 | 1.0 |
| Cpd (Ic) | 2 | 10.2 +/− 1.3 | 1.1 |
| | 0.2 | 8.8 +/− 0.5 | 1.0 |
| | 0.02 | 8.2 +/− 0.5 | 0.9 |

^{a}Fold stimulation over palmitate oxidation rate in the absence of test compounds

To determine the EC₅₀ value for the most potent compound (Ia) and to compare it to the effect of TOFA on palmitate oxidation in this cellular system, the dose response test with L6 cells was repeated at lower concentrations (0.2, 0.02 and 0.002 μM for compound (Ia) and 25, 2.5 and 0.25 μM for TOFA). The results are summarized in Table 3. The EC₅₀ for Compound (Ia) was found to lie between 0.002 μM and 0.02 μM, a result that emphasizes the high potency of this structural entity. TOFA also stimulated palmitate oxidation (maximally 1.5-fold, see Table 3) though it did not lead to the same level of activation as that induced by compound (Ia) even at the highest concentration tested (25 μM).

TABLE 3
Potency of compound (Ia) and TOFA to stimulate palmitate
oxidation rate in differentiated L6 cells

| | | Palmitate oxidation rate | Fold |
|---|---|---|---|
| Compound | Conc. (μM) | (pmol · h⁻¹ · well⁻¹) | stimulation^{a} |
| — | — | 5.4 +/− 0.7 | — |
| Cpd (Ia) | 0.2 | 14.6 +/− 0.1 | 2.7 |
| | 0.02 | 12.1 +/− 1.0 | 2.2 |
| | 0.002 | 8.3 +/− 0.0 | 1.5 |
| TOFA | 25 | 7.2 +/− 0.3 | 1.3 |
| | 2.5 | 8.0 +/− 0.7 | 1.5 |
| | 0.25 | 5.8 +/− 0.4 | 1.1 |

^{a}Fold stimulation over palmitate oxidation rate in the absence of test compounds

Stimulation of fatty acid oxidation in differentiated human muscle cells can be measured in analogy to the experiments described above. Differentiated human muscle cells are commercially available from PromoCell GmbH, D-69120 Heidelberg, Germany. In vivo assessment of fatty acid oxidation and lipid utilization

Compound (Ia) was tested in normal Wistar rats by two different methods to assess in vivo a) direct effects on fatty acid oxidation by following the conversion of [¹⁴C]-labeled palmitate to ¹⁴CO₂ exhaled by the animals and b) effects on total lipid utilization as determined by indirect calorimetry.

a) In vivo oxidation of [U-¹⁴C]palmitate. Male Wistar rats (240-360 g) were given chow food ad libitum. The day before the experiment each animal was singly caged and food was withdrawn 2.5 hours before the experiment. The animals were given [U-¹⁴C]palmitate (5 μCi) dissolved in 500 μl of olive oil by gavage and compound (Ia) (30 mg/kg) or saline (vehicle) by subcutaneous injection. Immediately after receiving [U-¹⁴C]palmitate and compound (Ia) or vehicle, the animals were kept singly in a metabolic chamber during six hours. The chamber was set up to allow quantive recovery of ¹⁴CO₂ exhaled by the animals. The chamber consisted of a desiccator with an elevated platform for the rat, an inlet for air at the bottom wall of the vessel. The flow of the air-exchange was controlled by a flow meter connected to a vacuum pump and set to 50 litres/hour. The temperature inside the chamber was kept at 22° C. Exhaled ¹⁴CO₂ from the animals was absorbed by a flask containing 500 ml of Carbosorb E (Canberra Packard). For each time-point 2.5 ml of Carbosorb E was withdrawn and mixed with 12.5 ml Ultima Flo (Canberra Packard) and ¹⁴C was deterimined by scintillation measurement in a Tri-Carb liquid scintillation counter (Canberra Packard). As shown in Table 4, compound (Ia) significantly increased the rate of palmitate oxidation between 40 and 77%; the effect being apparent approx. 60 min. after administration and lasting for the entire assessment period (6 hours).

TABLE 4
Stimulation of palmitate oxidation rate by compound (Ia)
in Wistar rats (n = 4)

| | Accumulated ¹⁴CO₂ exhaled (cpm) | | |
|---|---|---|---|
| Time | | Compound (Ia), | |
| (min) | Vehicle | 30 mg/kg | % increase over vehicle |
| 5 | 157 +/− 52 | 133 +/− 41 | −15 |
| 15 | 157 +/− 52 | 121 +/− 36 | −30 |
| 30 | 181 +/− 73 | 211 +/− 32 | 17 |
| 60 | 385 +/− 55 | 544 +/− 102 | 41 |
| 90 | 1028 +/− 75 | 1440 +/− 90 | 40 |
| 120 | 1680 +/− 121 | 2614 +/− 136 | 56 |
| 150 | 2677 +/− 202 | 4333 +/− 235 | 62 |
| 180 | 3787 +/− 155 | 5729 +/− 397 | 51 |
| 240 | 6133 +/− 398 | 9572 +/− 809 | 56 |
| 300 | 7508 +/− 422 | 13261 +/− 1.445 | 77 |
| 360 | 10099 +/− 1.181 | 16134 +/− 2.130 | 60 |

b) Indirect calorimetry. Substrate utilization can be measured indirectly from respiratory gas exchanges. The method is based on the measurement of oxygen (O₂) consumption and carbon-dioxide (CO₂) release, both originating from the oxidation of the energetic substrates that progressively release the chemical energy stored in the carbon-hydrogen bonds of carbohydrates, lipids (and proteins). The amounts of carbohydrate and lipid being oxidized in the body at any given time can be calculated from the volumes of 02 consumption and CO₂ exhalation by laboratory animals (and humans) in metabolic chambers. For a review of the methodology and the theoretical basis of indirect calorimetry, see Ferrannini, Metabolism, 37, 287-301, 1988.

An established, commercially available metabolic chamber apparatus (Oxymax, columbus Instruments, Ohio) was used to determine the gas exchange of vehicle-treated and compound (Ia)-treated Wistar rats in order to make precise and noninvasive measurements of the changes in the nature of the energetic substrate being oxidized by Wistar rats. Table 5 summarizes the data of the calculated lipid utilization determined for animals treated with two doses of compound (Ia), 10 and 30 mg/kg, administered subcutaneously 30 min. before starting the gas exchange measurements.

TABLE 5
Stimulation of lipid oxidation rate by compound (Ia) in Wistar rats (n = 8)
as determined by indirect calorimetry

| | Accumulated lipid utilization (g/kg body weight) | | | | |
|---|---|---|---|---|---|
| | | Compound (Ia), | % increase | Compound (Ia), | % increase |
| Time (min) | Vehicle | (10 mg/kg) | over vehicle | (30 mg/kg) | over vehicle |
| 33.5 | 0.21 | 0.24 | 12.6 | 0.27 | 28.4 |
| 67 | 0.45 | 0.49 | 9.6 | 0.54 | 19.2 |
| 100.5 | 0.69 | 0.76 | 9.8 | 0.79 | 14.5 |
| 134 | 0.93 | 1.03 | 9.8 | 1.06 | 13.2 |
| 167.5 | 1.20 | 1.31 | 9.2 | 1.35 | 12.5 |
| 201 | 1.48 | 1.61 | 8.4 | 1.66 | 11.8 |
| 234.5 | 1.76 | 1.89 | 7.8 | 1.96 | 11.7 |
| 268 | 2.01 | 2.17 | 7.9 | 2.26 | 12.2 |
| 301.5 | 2.27 | 2.44 | 7.7 | 2.55 | 12.5 |
| 335 | 2.55 | 2.73 | 7.2 | 2.86 | 12.3 |
| 368.5 | 2.85 | 3.03 | 6.3 | 3.18 | 11.5 |

Compound (Ia) dose-dependently stimulated total lipid oxidation by 6 to 12% (at 10 mg/kg) and 11 to 28% (at 30 mg/kg). The effect of compound (Ia) lasted for at least 6 hours. Although the stimulatory effect of compound (Ia) on total lipid utilization is less pronounced than that on utilization of free fatty acids, it is of significant magnitude and represents a substantial increase of body fat mobilization and oxidation.

The compounds of formula I and their pharmaceutically acceptable esters can be used as medicaments, e.g. in the form of pharmaceutical preparations for enteral, parenteral or topical administration. They can be administered, for example, perorally, e.g. in the form of tablets, coated tablets, dragées, hard and soft gelatine capsules, solutions, emulsions or suspensions, rectally, e.g. in the form of suppositories, parenterally, e.g. in the form of injection solutions or infusion solutions, or topically, e.g. in the form of ointments, creams or oils. Oral administration is preferred.

The production of the pharmaceutical preparations can be effected in a manner which will be familiar to any person skilled in the art by bringing the described compounds of formula I and their pharmaceutically acceptable esters, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragées and hard gelatine capsules. Suitable carrier materials for soft gelatine capsules are, for example, vegetable oils, waxes, fats and semi-solid and liquid polyols (depending on the nature of the active ingredient no carriers might, however, be required in the case of soft gelatine capsules). Suitable carrier materials for the production of solutions and syrups are, for example, water, polyols, sucrose, invert sugar and the like. Suitable carrier materials for injection solutions are, for example, water, alcohols, polyols, glycerol and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavour-improving agents, salts for varying the osmotic pressure, buffer substances, solubilizers, colorants and masking agents and antioxidants come into consideration as pharmaceutical adjuvants.

The dosage of the compounds of formula I can vary within wide limits depending on the disease to be controlled, the age and the individual condition of the patient and the mode of administration, and will, of course, be fitted to the individual requirements in each particular case. For adult patients a daily dosage of about 1 to 100 mg, especially about 1 to 10 mg, comes into consideration in context with the diseases mentioned above. Depending on severity of the disease and the precise pharmacokinetic profile the compound could be administered with one or several daily dosage units, e.g. in 1 to 3 dosage units.

The pharmaceutical preparations conveniently contain about 1-100 mg, preferably 1-10 mg, of a compound of formula I.

The following Examples serve to illustrate the present invention in more detail. They are, however, not intended to limit its scope in any manner.

### EXAMPLES

### Example A

Film coated tablets containing the following ingredients can be manufactured in a conventional manner:

| | |
|---|---|
| Ingredients | Per tablet |

| | | | | |
|---|---|---|---|---|
| Kernel: | | | | |
| Compound of formula (I) | 10.0 | mg | 200.0 | mg |
| Microcrystalline cellulose | 23.5 | mg | 43.5 | mg |
| Lactose hydrous | 60.0 | mg | 70.0 | mg |
| Povidone K30 | 12.5 | mg | 15.0 | mg |
| Sodium starch glycolate | 12.5 | mg | 17.0 | mg |
| Magnesium stearate | 1.5 | mg | 4.5 | mg |
| (Kernel Weight) | 120.0 | mg | 350.0 | mg |
| Film Coat: | | | | |
| Hydroxypropyl methyl cellulose | 3.5 | mg | 7.0 | mg |
| Polyethylene glycol 6000 | 0.8 | mg | 1.6 | mg |
| Talc | 1.3 | mg | 2.6 | mg |
| Iron oxyde (yellow) | 0.8 | mg | 1.6 | mg |
| Titan dioxide | 0.8 | mg | 1.6 | mg |

The active ingredient is sieved and mixed with microcristalline cellulose and the mixture is granulated with a solution of polyvinylpyrrolidon in water. The granulate is mixed with sodium starch glycolate and magesiumstearate and compressed to yield kernels of 120 or 350 mg respectively. The kernels are lacquered with an aqueous solution/suspension of the above mentioned film coat.

### Example B

Capsules containing the following ingredients can be manufactured in a conventional manner:

| | |
|---|---|
| Ingredients | Per capsule |

| | | |
|---|---|---|
| Compound of formula (I) | 25.0 | mg |
| Lactose | 150.0 | mg |
| Maize starch | 20.0 | mg |
| Talc | 5.0 | mg |

The components are sieved and mixed and filled into capsules of size 2.

### Example C

Injection solutions can have the following composition:

| | |
|---|---|
| Compound of formula (I) | 3.0 mg |
| Polyethylene Glycol 400 | 150.0 mg |
| Acetic Acid | q.s. ad pH 5.0 |
| Water for injection solutions | ad 1.0 ml |

The active ingredient is solved in a mixture of Polyethylene Glycol 400 and water for injection (part). The pH is adjusted to 5.0 by Acetic Acid. The volume is adjusted to 1.0 ml by addition of the residual amount of water. The solution is filtered, filled into vials using an appropriate overage and sterilized.

### Example D

Soft gelatin capsules containing the following ingredients can be manufactured in a conventional manner:

Capsule contents

| | |
|---|---|
| Compound of formula (I) | 5.0 mg |
| Yellow wax | 8.0 mg |
| Hydrogenated Soya bean oil | 8.0 mg |
| Partially hydrogenated plant oils | 34.0 mg |
| Soya bean oil | 110.0 mg |
| Weight of capsule contents | 165.0 mg |
| Gelatin capsule | |
| Gelatin | 75.0 mg |
| Glycerol 85% | 32.0 mg |
| Karion 83 | 8.0 mg (dry matter) |
| Titan dioxide | 0.4 mg |
| Iron oxide yellow | 1.1 mg |

The active ingredient is solved in a warm melting of the other ingredients and the mixture is filled into soft gelatin capsules of appropriate size. The filled soft gelatin capsules are treated according to the usual procedures.

### Example E

Sachets containing the following ingredients can be manufactured in a conventional manner:

| | | |
|---|---|---|
| Compound of formula (I) | 50.0 | mg |
| Lactose, fine powder | 1015.0 | mg |
| Microcristalline cellulose (AVICEL PH 102) | 1400.0 | mg |
| Sodium carboxymethyl cellulose | 14.0 | mg |
| Polyvinylpyrrolidon K 30 | 10.0 | mg |
| Magnesiumstearate | 10.0 | mg |
| Flavoring additives | 1.0 | mg |

The active ingredient is mixed with lactose, microcristalline cellulose and Sodium carboxymethyl cellulose and granulated with a mixture of polyvinylpyrrolidon in water. The granulate is mixed with magnesiumstearate and the flavouring additives and filled into sachets.

1. A pharmaceutical composition for the treatment or prophylaxis of diseases associated with ACCβ activity, fatty acid oxidation or both comprising a compound of the formula 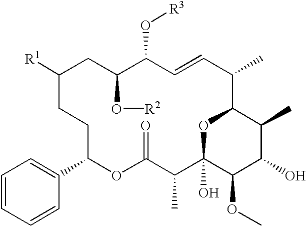
wherein
R¹ is hydrogen or hydroxy,
R² is hydrogen or lower-alkyl,
R³ is hydrogen or lower-alkyl,
and pharmaceutically acceptable esters thereof,
and at least one pharmaceutically acceptable carrier or adjuvant or combinations thereof.

2. The pharmaceutical composition of claim 1 wherein the compound is a compound of the formula 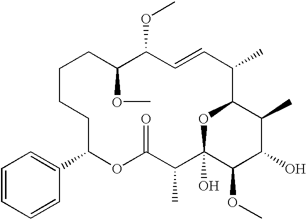
and pharmaceutically acceptable esters thereof.

3. The pharmaceutical composition of claim 1 wherein the compound is a compound of the formula 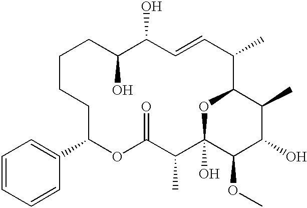
and pharmaceutically acceptable esters thereof.

4. The pharmaceutical composition of claim 1 wherein the compound is a compound of the formula 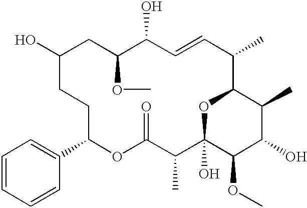
and pharmaceutically acceptable esters thereof.

5. A method for the treatment or prophylaxis of a disease associated with ACCβ activity, fatty acid oxidation or both, which method comprises administering to a human being or other animal a pharmaceutically effective amount of a compound of the formula 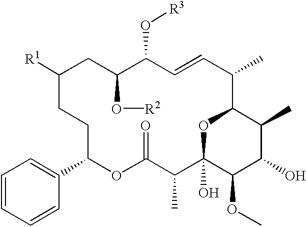
wherein
R¹ is hydrogen or hydroxy,
R² is hydrogen or lower-alkyl,
R³ is hydrogen or lower-alkyl,
and pharmaceutically acceptable esters thereof.

6. The method according to claim 5, wherein the disease is diabetes.

7. The method according to claim 5, wherein the disease is non insulin dependent diabetes mellitus.

8. The method according to claim 5, wherein the disease is obesity.

9. The method according to claim 5, wherein the disease is dyslipidemia.

10. The method according to claim 5 wherein the compound is a compound of the formula 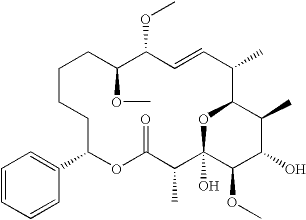
and pharmaceutically acceptable esters thereof.

11. The method according to claim 10 wherein the disease is diabetes, obesity or dislipidemia.

12. The method according to claim 5 wherein the compound is a compound of the formula 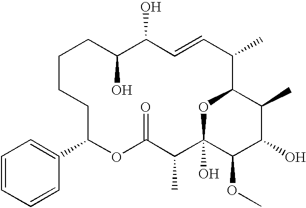
and pharmaceutically acceptable esters thereof.

13. The method according to claim 12 wherein the disease is diabetes, obesity or dislipidemia.

14. The method according to claim 5 wherein the compound is a compound of the formula 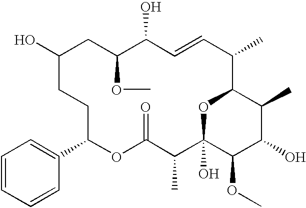
and pharmaceutically acceptable esters thereof.

15. The method according to claim 14 wherein the disease is diabetes, obesity or dyslipidemia.
